(19) United States
(12) Patent Application Publication
(10) Pub. No.: US 2005/0059897 A1
(43) Pub. Date: Mar. 17, 2005

(54) Statistical analysis for implantable cardiac devices

(76) Inventors: Jeffery D. Snell, Chatsworth, CA (US); Gene A. Bornzin, Simi Valley, CA (US)

Correspondence Address: PACESETTER, INC., 15900 VALLEY VIEW COURT, SYLMAR, CA, 91392-9221, US

(21) Appl. No.: 10/665,317

(22) Filed: Sep. 17, 2003

(51) Int. Cl. A61B 5/0402

(52) U.S. Cl. 600/510

(57) ABSTRACT

Exemplary methods and devices for analyzing intracardiac electrocardiograms (IEGMs) using statistical analysis (e.g., histograms, etc.), ensemble averaging and/or an ensemble average. Various methods and/or devices are suitable for use with atrial and/or ventricular pacing therapies. Other methods, devices and/or systems are also disclosed.

### CROSS-REFERENCE TO RELATED APPLICATIONS

This application is related to copending U.S. patent application Ser. No. ______, titled “Ensemble Averaging for Evoked Responses” (Attorney Docket No. A03P1063), filed concurrently herewith.

### TECHNICAL FIELD

Exemplary methods and/or devices presented herein generally relate to cardiac pacing and/or stimulation therapy. Various exemplary methods and/or devices concern statistical analysis, ensemble averaging and/or other techniques for analyzing cardiac information.

### BACKGROUND

Many implantable pacing devices rely on pacing schemes that sense atrial and/or ventricular activity. Often, to determine whether a particular cardiac event has occurred, a pacing device analyzes sensed information using threshold and/or derivative techniques. For example, sensed information having a relatively abrupt change in amplitude may, upon analysis, indicate occurrence of an R wave. In this example, the change in amplitude is amenable to analysis by a threshold technique (e.g., through use of a threshold amplitude) and/or a derivative technique (e.g., analysis of amplitude with respect to time). In general, such analyses operate in a binary manner, i.e., indicating that an event occurred or that an event did not occur. Thus, an implantable pacing device typically gleans little knowledge about underlying cardiac behavior when operated in such a manner.

Various exemplary methods and/or devices described herein enhance detection and/or characterization of cardiac activity. In particular, such methods and/or devices address signal-to-noise, electrode polarization and/or other issues related to detection and/or characterization of cardiac activity.

### SUMMARY

Exemplary methods and devices are disclosed herein for analyzing intracardiac electrocardiograms (IEGMs) using statistical analysis (e.g., histograms, etc.), ensemble averaging and/or an ensemble average. Various methods and/or devices are suitable for use with atrial and/or ventricular pacing therapies. In general, the various exemplary devices and methods described herein, and equivalents thereof, are suitable for use in a variety of pacing therapies and/or other cardiac related therapies.

### BRIEF DESCRIPTION OF THE DRAWINGS

Features and advantages of the described implementations can be more readily understood by reference to the following description taken in conjunction with the accompanying drawings.

FIG. 1 is a simplified diagram illustrating an exemplary implantable stimulation device in electrical communication with at least three leads implanted into a patient's heart and at least one other lead for delivering stimulation and/or shock therapy.

FIG. 2 is a functional block diagram of an exemplary implantable stimulation device illustrating basic elements that are configured to provide cardioversion, defibrillation, pacing stimulation and/or autonomic nerve stimulation or other tissue and/or nerve stimulation. The implantable stimulation device is further configured to sense information and administer stimulation pulses responsive to such information.

FIG. 3 is a diagram of exemplary waveforms that exhibit cardiac activity responsive to native and/or applied stimuli.

FIG. 4 is a diagram of various exemplary atrial waveforms including native (P wave), capture (A wave), noncapture (with atrial stimulus), atrial fusion (P wave and A wave complex) and atrial pseudofusion (P wave with atrial stimulus).

FIG. 5 is a diagram of exemplary ventricular information including a ventricular stimulus, ventricular afterpotential and a ventricular IEGM.

FIG. 6 is a diagram of exemplary atrial information including an atrial stimulus, atrial afterpotential and an atrial IEGM.

FIG. 7 is a diagram of a series of exemplary atrial IEGMs and an exemplary ensemble average of the series of atrial IEGMs.

FIG. 8 is a diagram of a series of exemplary ventricular IEGMs and an exemplary ensemble average of the series of ventricular IEGMs.

FIG. 9 is a diagram of a series of exemplary native waveforms and an exemplary ensemble average of the series of native waveforms.

FIG. 10 is a diagram of an exemplary waveform and an exemplary histogram distribution for a set amplitude level.

FIG. 11 is a diagram of an exemplary waveform and an exemplary bimodal histogram distribution for a set amplitude level.

FIG. 12 is a diagram of an exemplary waveform and an exemplary histogram distribution for a set time.

FIG. 13 is a diagram of various exemplary histograms.

FIG. 14 is a diagram of an exemplary acquisition and analysis scheme for acquisition of atrial information including an atrial IEGM and atrial channels.

FIG. 15 is a block diagram of an exemplary method using histogram analysis.

FIG. 16 is a block diagram of an exemplary method using ensemble averaging and histogram analysis.

FIG. 17 is a block diagram of an exemplary method for analyzing sensed information.

FIG. 18 is a block diagram of an exemplary method for comparing to existing histograms, sets, etc.

FIG. 19 is a block diagram of an exemplary method for acquiring information and comparing acquired information.

### DETAILED DESCRIPTION

The following description is of the best mode presently contemplated for practicing the described implementations. This description is not to be taken in a limiting sense, but rather is made merely for the purpose of describing the general principles of the implementations. The scope of the described implementations should be ascertained with reference to the issued claims. In the description that follows, like numerals or reference designators will be used to reference like parts or elements throughout.

Exemplary Stimulation Device

The techniques described below are intended to be implemented in connection with any stimulation device that is configured or configurable to stimulate nerves and/or stimulate and/or shock a patient's heart.

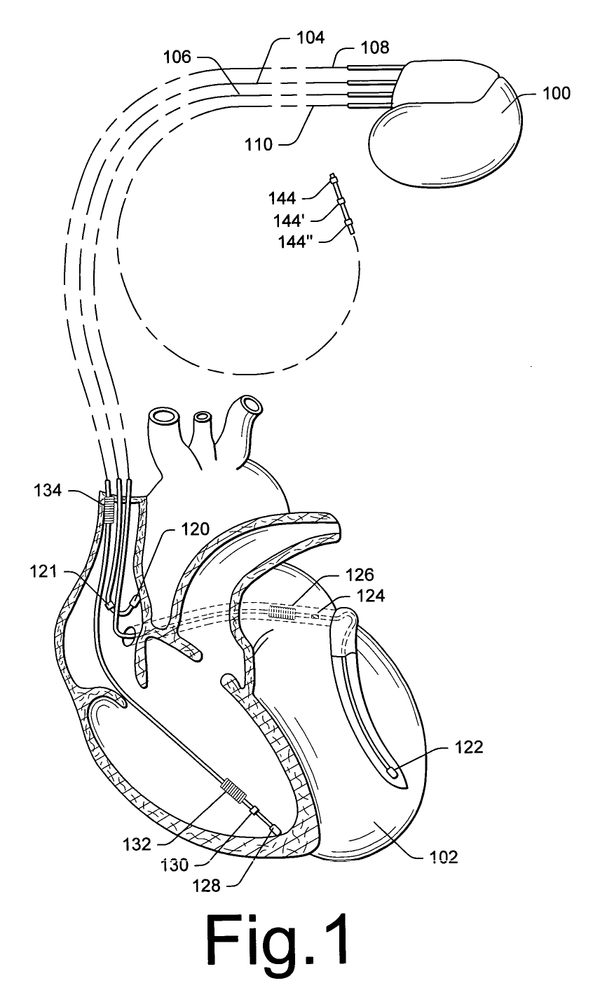

FIG. 1 shows an exemplary stimulation device 100 in electrical communication with a patient's heart 102 by way of three leads 104, 106, 108, suitable for delivering multi-chamber stimulation and shock therapy. The leads 104, 106, 108 are optionally configurable for delivery of stimulation pulses suitable for stimulation of autonomic nerves. In addition, the device 100 includes a fourth lead 110 having, in this implementation, three electrodes 144, 144′, 144″ suitable for stimulation of autonomic nerves. This lead may be positioned in and/or near a patient's heart or near an autonomic nerve within a patient's body and remote from the heart. The right atrial lead 104, as the name implies, is positioned in and/or passes through a patient's right atrium. The right atrial lead 104 optionally senses atrial cardiac signals and/or provide right atrial chamber stimulation therapy. As shown in FIG. 1, the stimulation device 100 is coupled to an implantable right atrial lead 104 having, for example, an atrial tip electrode 120, which typically is implanted in the patient's right atrial appendage. The lead 104, as shown in FIG. 1, also includes an atrial ring electrode 121. Of course, the lead 104 may have other electrodes as well. For example, the right atrial lead optionally includes a distal bifurcation having electrodes suitable for stimulation of autonomic nerves.

To sense atrial cardiac signals, ventricular cardiac signals and/or to provide chamber pacing therapy, particularly on the left side of a patient's heart, the stimulation device 100 is coupled to a coronary sinus lead 106 designed for placement in the coronary sinus and/or tributary veins of the coronary sinus. Thus, the coronary sinus lead 106 is optionally suitable for positioning at least one distal electrode adjacent to the left ventricle and/or additional electrode(s) adjacent to the left atrium. In a normal heart, tributary veins of the coronary sinus include, but may not be limited to, the great cardiac vein, the left marginal vein, the left posterior ventricular vein, the middle cardiac vein, and the small cardiac vein.

Accordingly, an exemplary coronary sinus lead 106 is optionally designed to receive atrial and ventricular cardiac signals and to deliver left ventricular pacing therapy using, for example, at least a left ventricular tip electrode 122, left atrial pacing therapy using at least a left atrial ring electrode 124, and shocking therapy using at least a left atrial coil electrode 126. For a complete description of a coronary sinus lead, the reader is directed to U.S. patent application Ser. No. 09/457,277, filed Dec. 8, 1999, entitled “A Self-Anchoring, Steerable Coronary Sinus Lead” (Pianca et al.; and U.S. Pat. No. 5,466,254, “Coronary Sinus Lead with Atrial Sensing Capability” (Helland), which are incorporated herein by reference. The coronary sinus lead 106 further optionally includes electrodes for stimulation of autonomic nerves. Such a lead may include pacing and autonomic nerve stimulation functionality and may further include bifurcations or legs. For example, an exemplary coronary sinus lead includes pacing electrodes capable of delivering pacing pulses to a patient's left ventricle and at least one electrode capable of stimulating an autonomic nerve. An exemplary coronary sinus lead (or left ventricular lead or left atrial lead) may also include at least one electrode capable of stimulating an autonomic nerve, such an electrode may be positioned on the lead or a bifurcation or leg of the lead.

Stimulation device 100 is also shown in electrical communication with the patient's heart 102 by way of an implantable right ventricular lead 108 having, in this exemplary implementation, a right ventricular tip electrode 128, a right ventricular ring electrode 130, a right ventricular (RV) coil electrode 132, and an SVC coil electrode 134. Typically, the right ventricular lead 108 is transvenously inserted into the heart 102 to place the right ventricular tip electrode 128 in the right ventricular apex so that the RV coil electrode 132 will be positioned in the right ventricle and the SVC coil electrode 134 will be positioned in the superior vena cava. Accordingly, the right ventricular lead 108 is capable of sensing or receiving cardiac signals, and delivering stimulation in the form of pacing and shock therapy to the right ventricle. An exemplary right ventricular lead may also include at least one electrode capable of stimulating an autonomic nerve, such an electrode may be positioned on the lead or a bifurcation or leg of the lead.

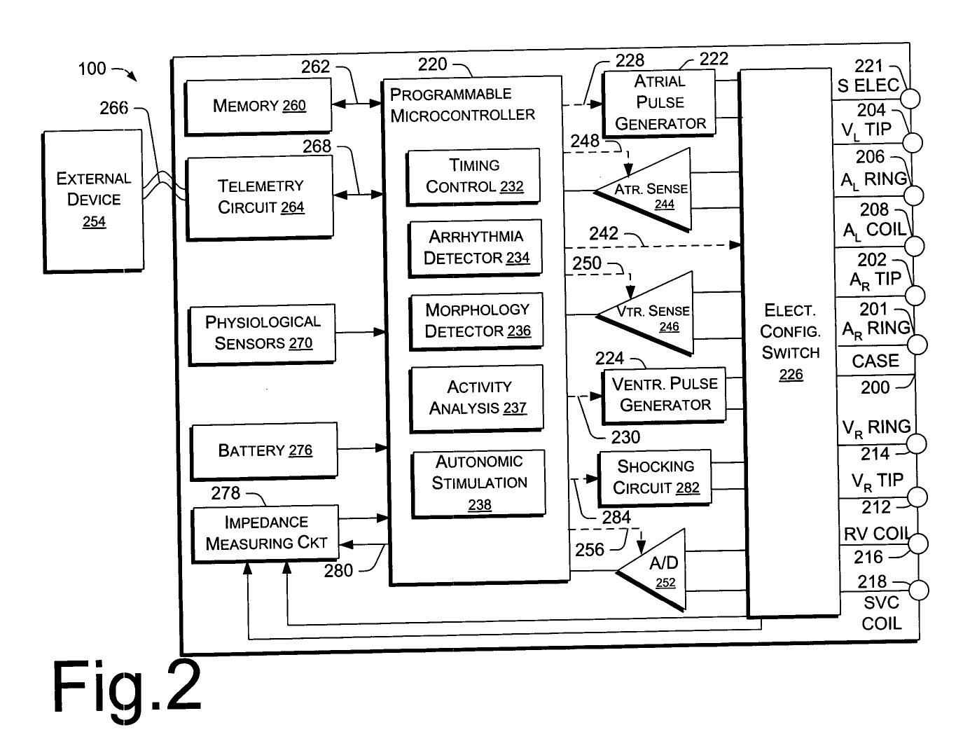

FIG. 2 shows an exemplary, simplified block diagram depicting various components of stimulation device 100. The stimulation device 100 can be capable of treating both fast and slow arrhythmias with stimulation therapy, including cardioversion, defibrillation, and pacing stimulation. The stimulation device can be solely or further capable of delivering stimuli to autonomic nerves. While a particular multi-chamber device is shown, it is to be appreciated and understood that this is done for illustration purposes only. Thus, the techniques and methods described below can be implemented in connection with any suitably configured or configurable stimulation device. Accordingly, one of skill in the art could readily duplicate, eliminate, or disable the appropriate circuitry in any desired combination to provide a device capable of treating the appropriate chamber(s) or regions of a patient's heart with cardioversion, defibrillation, pacing stimulation, and/or autonomic nerve stimulation.

Housing 200 for stimulation device 100 is often referred to as the “can”, “case” or “case electrode”, and may be programmably selected to act as the return electrode for all “unipolar” modes. Housing 200 may further be used as a return electrode alone or in combination with one or more of the coil electrodes 126, 132 and 134 for shocking purposes. Housing 200 further includes a connector (not shown) having a plurality of terminals 201, 202, 204, 206, 208, 212, 214, 216, 218, 221 (shown schematically and, for convenience, the names of the electrodes to which they are connected are shown next to the terminals).

To achieve right atrial sensing, pacing and/or autonomic stimulation, the connector includes at least a right atrial tip terminal (AR TIP) 202 adapted for connection to the atrial tip electrode 120. A right atrial ring terminal (A_{R} RING) 201 is also shown, which is adapted for connection to the atrial ring electrode 121. To achieve left chamber sensing, pacing, shocking, and/or autonomic stimulation, the connector includes at least a left ventricular tip terminal (V_{L} TIP) 204, a left atrial ring terminal (A_{L} RING) 206, and a left atrial shocking terminal (A_{L} COIL) 208, which are adapted for connection to the left ventricular tip electrode 122, the left atrial ring electrode 124, and the left atrial coil electrode 126, respectively. Connection to suitable autonomic nerve stimulation electrodes is also possible via these and/or other terminals (e.g., via a nerve stimulation terminal S ELEC 221).

To support right chamber sensing, pacing, shocking, and/or autonomic nerve stimulation, the connector further includes a right ventricular tip terminal (V_{R} TIP) 212, a right ventricular ring terminal (V_{R} RING) 214, a right ventricular shocking terminal (RV COIL) 216, and a superior vena cava shocking terminal (SVC COIL) 218, which are adapted for connection to the right ventricular tip electrode 128, right ventricular ring electrode 130, the RV coil electrode 132, and the SVC coil electrode 134, respectively. Connection to suitable autonomic nerve stimulation electrodes is also possible via these and/or other terminals (e.g., via the nerve stimulation terminal S ELEC 221).

At the core of the stimulation device 100 is a programmable microcontroller 220 that controls the various modes of stimulation therapy. As is well known in the art, microcontroller 220 typically includes a microprocessor, or equivalent control circuitry, designed specifically for controlling the delivery of stimulation therapy, and may further include RAM or ROM memory, logic and timing circuitry, state machine circuitry, and I/O circuitry. Typically, microcontroller 220 includes the ability to process or monitor input signals (data or information) as controlled by a program code stored in a designated block of memory. The type of microcontroller is not critical to the described implementations. Rather, any suitable microcontroller 220 may be used that carries out the functions described herein. The use of microprocessor-based control circuits for performing timing and data analysis functions are well known in the art.

Representative types of control circuitry that may be used in connection with the described embodiments can include the microprocessor-based control system of U.S. Pat. No. 4,940,052 (Mann et al.), the state-machine of U.S. Pat. No. 4,712,555 (Thornander et al.) and U.S. Pat. No. 4,944,298 (Sholder), all of which are incorporated by reference herein. For a more detailed description of the various timing intervals used within the stimulation device and their inter-relationship, see U.S. Pat. No. 4,788,980 (Mann et al.), also incorporated herein by reference.

FIG. 2 also shows an atrial pulse generator 222 and a ventricular pulse generator 224 that generate pacing stimulation pulses for delivery by the right atrial lead 104, the coronary sinus lead 106, and/or the right ventricular lead 108 via an electrode configuration switch 226. It is understood that in order to provide stimulation therapy in each of the four chambers of the heart (or to autonomic nerves) the atrial and ventricular pulse generators, 222 and 224, may include dedicated, independent pulse generators, multiplexed pulse generators, or shared pulse generators. The pulse generators 222 and 224 are controlled by the microcontroller 220 via appropriate control signals 228 and 230, respectively, to trigger or inhibit the stimulation pulses.

Microcontroller 220 further includes timing control circuitry 232 to control the timing of the stimulation pulses (e.g., pacing rate, atrio-ventricular (AV) delay, atrial interconduction (A-A) delay, or ventricular interconduction (V-V) delay, etc.) as well as to keep track of the timing of refractory periods, blanking intervals, noise detection windows, evoked response windows, alert intervals, marker channel timing, etc., which is well known in the art.

Microcontroller 220 further includes an arrhythmia detector 234, a morphology detector 236, and optionally an orthostatic compensator and a minute ventilation (MV) response module, the latter two are not shown in FIG. 2. These components can be utilized by the stimulation device 100 for determining desirable times to administer various therapies, including those to reduce the effects of orthostatic hypotension. The aforementioned components may be implemented in hardware as part of the microcontroller 220, or as software/firmware instructions programmed into the device and executed on the microcontroller 220 during certain modes of operation.

Microcontroller 220 further includes an activity analysis module 237. The activity analysis module 237 optionally implements one or more methods for sensing, information analysis, and/or stimulation control related to cardiac activity. For example, the activity analysis module 237 optionally implements one or more of the exemplary methods described below.

Microcontroller 220 further includes an autonomic nerve stimulation module 238 for performing a variety of tasks related to autonomic nerve stimulation. This component can be utilized by the stimulation device 100 for determining desirable times to administer various therapies, including, but not limited to, parasympathetic stimulation. The autonomic module 238 may be implemented in hardware as part of the microcontroller 220, or as software/firmware instructions programmed into the device and executed on the microcontroller 220 during certain modes of operation.

The electronic configuration switch 226 includes a plurality of switches for connecting the desired electrodes to the appropriate I/O circuits, thereby providing complete electrode programmability. Accordingly, switch 226, in response to a control signal 242 from the microcontroller 220, determines the polarity of the stimulation pulses (e.g., unipolar, bipolar, combipolar, etc.) by selectively closing the appropriate combination of switches (not shown) as is known in the art.

Atrial sensing circuits 244 and ventricular sensing circuits 246 may also be selectively coupled to the right atrial lead 104, coronary sinus lead 106, and the right ventricular lead 108, through the switch 226 for detecting the presence of cardiac activity in each of the four chambers of the heart. Accordingly, the atrial (ATR. SENSE) and ventricular (VTR. SENSE) sensing circuits, 244 and 246, may include dedicated sense amplifiers, multiplexed amplifiers, or shared amplifiers. Switch 226 determines the “sensing polarity” of the cardiac signal by selectively closing the appropriate switches, as is also known in the art. In this way, the clinician may program the sensing polarity independent of the stimulation polarity. The sensing circuits (e.g., 244 and 246) are optionally capable of obtaining information indicative of tissue capture.

Each sensing circuit 244 and 246 preferably employs one or automatic gain control, bandpass filtering, and a threshold detection circuit, as known in the art, to selectively sense the cardiac signal of interest. The automatic gain control enables the device 100 to deal effectively with the difficult problem of sensing the low amplitude signal characteristics of atrial or ventricular fibrillation.

The outputs of the atrial and ventricular sensing circuits 244 and 246 are connected to the microcontroller 220, which, in turn, is able to trigger or inhibit the atrial and ventricular pulse generators 222 and 224, respectively, in a demand fashion in response to the absence or presence of cardiac activity in the appropriate chambers of the heart. Furthermore, as described herein, the microcontroller 220 is also capable of analyzing information output from the sensing circuits 244 and 246 and/or the data acquisition system 252 to determine or detect whether and to what degree tissue capture has occurred and to program a pulse, or pulses, in response to such determinations. The sensing circuits 244 and 246, in turn, receive control signals over signal lines 248 and 250 from the microcontroller 220 for purposes of controlling the gain, threshold, polarization charge removal circuitry (not shown), and the timing of any blocking circuitry (not shown) coupled to the inputs of the sensing circuits, 244 and 246, as is known in the art.

For arrhythmia detection, the device 100 utilizes the atrial and ventricular sensing circuits, 244 and 246, to sense cardiac signals to determine whether a rhythm is physiologic or pathologic. In reference to arrhythmias, as used herein, “sensing” is reserved for the noting of an electrical signal or obtaining data (information), and “detection” is the processing (analysis) of these sensed signals and noting the presence of an arrhythmia. The timing intervals between sensed events (e.g., P-waves, R-waves, and depolarization signals associated with fibrillation which are sometimes referred to as “F-waves” or “Fib-waves”) are then classified by the arrhythmia detector 234 of the microcontroller 220 by comparing them to a predefined rate zone limit (i.e., bradycardia, normal, low rate VT. high rate VT. and fibrillation rate zones) and various other characteristics (e.g., sudden onset, stability, physiologic sensors, and morphology, etc.) in order to determine the type of remedial therapy that is needed (e.g., bradycardia pacing, anti-tachycardia pacing, cardioversion shocks or defibrillation shocks, collectively referred to as “tiered therapy”).

Cardiac signals are also applied to inputs of an analog-to-digital (A/D) data acquisition system 252. The data acquisition system 252 is configured to acquire intracardiac electrogram signals, convert the raw analog data into a digital signal, and store the digital signals for later processing and/or telemetric transmission to an external device 254. The data acquisition system 252 is coupled to the right atrial lead 104, the coronary sinus lead 106, the right ventricular lead 108 and/or the nerve stimulation lead through the switch 226 to sample cardiac signals across any pair of desired electrodes.

The microcontroller 220 is further coupled to a memory 260 by a suitable data/address bus 262, wherein the programmable operating parameters used by the microcontroller 220 are stored and modified, as required, in order to customize the operation of the stimulation device 100 to suit the needs of a particular patient. Such operating parameters define, for example, pacing pulse amplitude, pulse duration, electrode polarity, rate, sensitivity, automatic features, arrhythmia detection criteria, and the amplitude, waveshape, number of pulses, and vector of each shocking pulse to be delivered to the patient's heart 102 within each respective tier of therapy. One feature of the described embodiments is the ability to sense and store a relatively large amount of data (e.g., from the data acquisition system 252), which data may then be used for subsequent analysis to guide the programming of the device.

Advantageously, the operating parameters of the implantable device 100 may be non-invasively programmed into the memory 260 through a telemetry circuit 264 in telemetric communication via communication link 266 with the external device 254, such as a programmer, transtelephonic transceiver, or diagnostic system analyzer The microcontroller 220 activates the telemetry circuit 264 with a control signal 268. The telemetry circuit 264 advantageously allows intracardiac electrograms and status information relating to the operation of the device 100 (as contained in the microcontroller 220 or memory 260) to be sent to the external device 254 through an established communication link 266.

The stimulation device 100 can further include a physiologic sensor 270, commonly referred to as a “rate-responsive” sensor because it is typically used to adjust pacing stimulation rate according to the exercise state of the patient. However, the physiological sensor 270 may further be used to detect changes in cardiac output (see, e.g., U.S. Pat. No. 6,314,323, entitled “Heart stimulator determining cardiac output, by measuring the systolic pressure, for controlling the stimulation”, to Ekwall, issued Nov. 6, 2001, which discusses a pressure sensor adapted to sense pressure in a right ventricle and to generate an electrical pressure signal corresponding to the sensed pressure, an integrator supplied with the pressure signal which integrates the pressure signal between a start time and a stop time to produce an integration result that corresponds to cardiac output), changes in the physiological condition of the heart, or diurnal changes in activity (e.g., detecting sleep and wake states). Accordingly, the microcontroller 220 responds by adjusting the various pacing parameters (such as rate, AV Delay, V-V Delay, etc.) at which the atrial and ventricular pulse generators, 222 and 224, generate stimulation pulses.

While shown as being included within the stimulation device 100, it is to be understood that the physiologic sensor 270 may also be external to the stimulation device 100, yet still be implanted within or carried by the patient. Examples of physiologic sensors that may be implemented in device 100 include known sensors that, for example, sense respiration rate, pH of blood, ventricular gradient, cardiac output, preload, afterload, contractility, and so forth. Another sensor that may be used is one that detects activity variance, wherein an activity sensor is monitored diurnally to detect the low variance in the measurement corresponding to the sleep state. For a complete description of the activity variance sensor, the reader is directed to U.S. Pat. No. 5,476,483 (Bornzin et al.), issued Dec. 19, 1995, which patent is hereby incorporated by reference.

More specifically, the physiological sensors 270 optionally include sensors for detecting movement and minute ventilation in the patient. The physiological sensors 270 may include a position sensor and/or a minute ventilation (MV) sensor to sense minute ventilation, which is defined as the total volume of air that moves in and out of a patient's lungs in a minute. Signals generated by the position sensor and MV sensor are passed to the microcontroller 220 for analysis in determining whether to adjust the pacing rate, etc. The microcontroller 220 monitors the signals for indications of the patient's position and activity status, such as whether the patient is climbing upstairs or descending downstairs or whether the patient is sitting up after lying down.

The stimulation device additionally includes a battery 276 that provides operating power to all of the circuits shown in FIG. 2. For the stimulation device 100, which employs shocking therapy, the battery 276 is capable of operating at low current drains for long periods of time (e.g., preferably less than 10 μA), and is capable of providing high-current pulses (for capacitor charging) when the patient requires a shock pulse (e.g., preferably, in excess of 2 A, at voltages above 2 V, for periods of 10 seconds or more). The battery 276 also desirably has a predictable discharge characteristic so that elective replacement time can be detected.

The stimulation device 100 can further include magnet detection circuitry (not shown), coupled to the microcontroller 220, to detect when a magnet is placed over the stimulation device 100. A magnet may be used by a clinician to perform various test functions of the stimulation device 100 and/or to signal the microcontroller 220 that the external programmer 254 is in place to receive or transmit data to the microcontroller 220 through the telemetry circuits 264.

The stimulation device 100 further includes an impedance measuring circuit 278 that is enabled by the microcontroller 220 via a control signal 280. The known uses for an impedance measuring circuit 278 include, but are not limited to, lead impedance surveillance during the acute and chronic phases for proper lead positioning or dislodgement; detecting operable electrodes and automatically switching to an operable pair if dislodgement occurs; measuring respiration or minute ventilation; measuring thoracic impedance for determining shock thresholds; detecting when the device has been implanted; measuring stroke volume; and detecting the opening of heart valves, etc. The impedance measuring circuit 278 is advantageously coupled to the switch 226 so that any desired electrode may be used.

In the case where the stimulation device 100 is intended to operate as an implantable cardioverter/defibrillator (ICD) device, it detects the occurrence of an arrhythmia, and automatically applies an appropriate therapy to the heart aimed at terminating the detected arrhythmia. To this end, the microcontroller 220 further controls a shocking circuit 282 by way of a control signal 284. The shocking circuit 282 generates shocking pulses of low (up to 0.5 J), moderate (0.5 J to 10 J), or high energy (11 J to 40 J), as controlled by the microcontroller 220. Such shocking pulses are applied to the patient's heart 102 through at least two shocking electrodes, and as shown in this embodiment, selected from the left atrial coil electrode 126, the RV coil electrode 132, and/or the SVC coil electrode 134. As noted above, the housing 200 may act as an active electrode in combination with the RV electrode 132, or as part of a split electrical vector using the SVC coil electrode 134 or the left atrial coil electrode 126 (i.e., using the RV electrode as a common electrode).

Cardioversion level shocks are generally considered to be of low to moderate energy level (so as to minimize pain felt by the patient), and/or synchronized with an R-wave and/or pertaining to the treatment of tachycardia. Defibrillation shocks are generally of moderate to high energy level (i.e., corresponding to thresholds in the range of 5 J to 40 J), delivered asynchronously (since R-waves may be too disorganized), and pertaining exclusively to the treatment of fibrillation. Accordingly, the microcontroller 220 is capable of controlling the synchronous or asynchronous delivery of the shocking pulses.

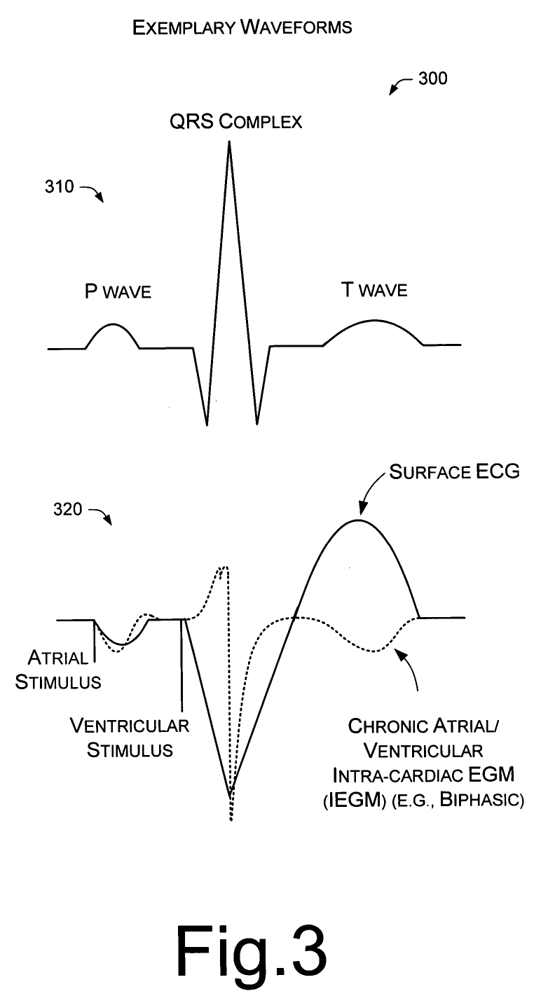

Referring to FIG. 3, exemplary waveforms 300 are shown. The waveforms 300 include an exemplary waveform 310 based on native cardiac activity and an exemplary ECG waveform 320 and an exemplary IEGM waveform 324 based on delivered stimuli. These exemplary waveforms 300 exhibit differences between native activity, paced activity and surface and intra-cardiac sensing. The IEGM 324 is representative of the type of waveform an implantable pacing device may sense. Also note the waveforms 320 and 324 show an atrial stimulus and a ventricular stimulus and no native stimuli; thus, these waveforms do not exhibit fusion and/or pseudofusion.

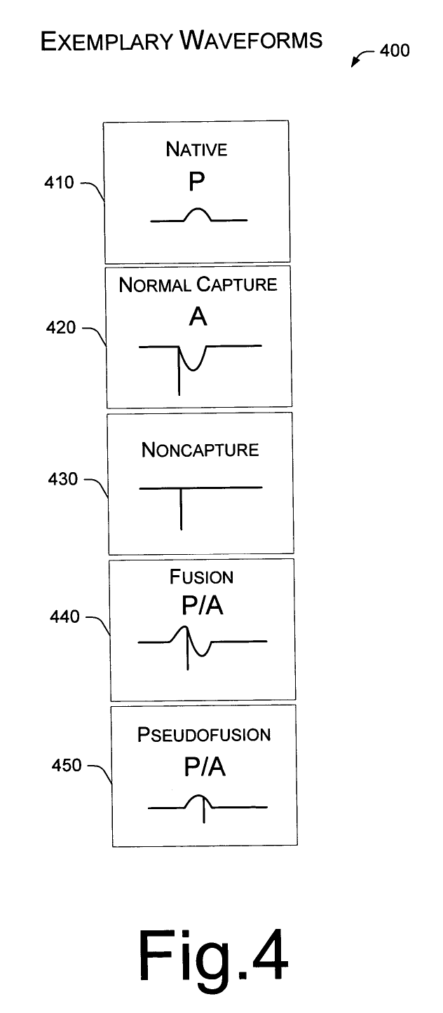

Referring to FIG. 4, various exemplary atrial waveforms 400 are shown. As discussed herein, an atrial waveform caused by an atrial stimulus is generally referred to as an “A wave” while an atrial waveform caused by a native stimulus (e.g., initiated by the sinoatrial node, etc.) is generally referred to as a “P wave”. A native P wave 410 may indicate that a patient does not need atrial pacing. However, if the native rate (e.g., as measured from P wave to P wave), exceeds or falls below a desirable rate, then atrial pacing may be desirable. An A wave 420 indicates that an applied atrial stimulus had sufficient power to “capture” atrial tissue. In comparison to the A wave 420, the noncapture waveform 430, indicates that the applied atrial stimulus did not have sufficient power to sufficiently capture atrial tissue. The last two waveforms 440, 450 exhibit atrial “fusion” and atrial “pseudofusion”, respectively.

Fusion is typically characterized by a wave complex formed by depolarization of the myocardium initiated by both a non-native stimulus and a native stimulus. Thus, atrial fusion is characterized by a wave complex (e.g., the waveform 440) initiated by a native stimulus and complex formed by depolarization of the myocardium initiated by a native stimulus; however, a non-native stimulus, that does not significantly contribute to depolarization, is present that distorts the wave complex. Thus, atrial pseudofusion is characterized by a wave complex (e.g., the waveform 450) initiated by a native stimulus and distorted by a paced atrial stimulus.

As demonstrated by these exemplary waveforms 400, for a variety of reasons, pacing devices that aim to detect and/or characterize cardiac activity may misinterpret fusion and/or pseudofusion waveforms as noncapture or loss of capture. Further, a pacing device may not need to implement pacing if a native stimulus is present. Again, in both fusion and pseudofusion (e.g., the waveforms 440, 450), native activity is present.

While the exemplary waveforms 400 generally pertain to atrial waveforms, similar waveforms exist that correspond to ventricles. In the description that follows, waveforms that correspond to native cardiac activity, ventricular evoked responses (e.g., ventricular capture or V waves) and/or atrial evoked responses (e.g., atrial capture or A waves) are discussed in more detail with respect to a variety of exemplary methods and/or devices that aim to enhance detection and/or characterization of cardiac activity. Such exemplary methods and/or devices optionally enhance detection and/or characterization of various waves (e.g., P waves, R, waves, T waves, etc.).

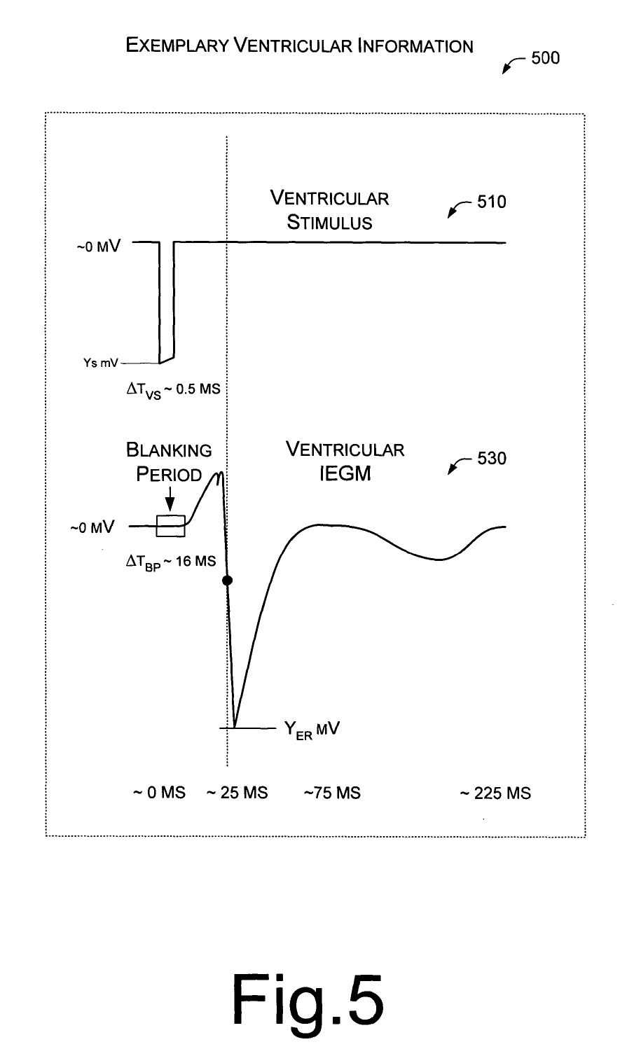

Referring to FIG. 5, exemplary ventricular information 500 is shown. The exemplary ventricular information 500 includes a plot of ventricular stimulus voltage versus time 510 and a plot of sensed activity with respect to time 530. The exemplary time scale in the plot 530 illustrates that ventricular activity typically occurs in a post-stimulus time frame of approximately one to several hundred milliseconds. Other events, such as the stimulation pulse width (e.g., Δtᵥₛ˜0.5 ms) of the plot 510, are also exemplary and may not correspond precisely to the time scale of the plot 530.

The plot 510 exhibits a stimulus at approximately 0 ms, wherein the stimulus has a magnitude of Yₛ mV and a duration of Δt ms. The ventricular IEGM plot 530 exhibits a ventricular stimulus at approximately 0 ms and a corresponding blanking period (e.g., Δt_{BP}˜16 ms). A blanking period is typically an interval initiated by the delivery of a stimulus during which sensing (e.g., a sense amplifier) is temporarily disabled. In dual-chamber pulse generators, a blanking period may also prevent inappropriate detection of signals from another chamber (e.g., crosstalk). Blanking periods are not available in all pacing devices and the blanking period, typically stated in milliseconds, may be preset or programmable.

As shown in FIG. 5, the IEGM in the plot 530 exhibits a minimum voltage, e.g., Y_{ER} mV, near approximately 25 ms. Just prior to this minimum, is a vertical dashed line and a solid dot that indicate one possible evoked response detection point. For example, a derivative-based evoked response detection scheme may detect a maximum negative derivative of voltage versus time at approximately the location of the dashed vertical line. In instances where a positive amplitude afterpotential exceeds the negative amplitude of ventricular depolarization, a ventricular IEGM may actually have positive amplitude. While such a hypothetical ventricular IEGM may, at such a neighborhood, have positive amplitude, it will typically maintain a negative slope. The presence of native activity may further complicate detection and/or characterization of “true” ventricular activity. Thus, as described herein, techniques that account for afterpotential and/or more thoroughly characterize waveforms help in detection and/or characterization of cardiac activity.

In general, ventricular activity differs from atrial activity in that intracardiac ventricular activity typically exhibits greater amplitude than intracardiac atrial activity. Further, the frequency response of intracardiac ventricular evoked responses is usually less than that of intracardiac atrial evoked responses. Yet further, atrial evoked responses typically occur more quickly following an atrial stimulus when compared to ventricular evoked responses following a ventricular stimulus. Thus, for implantable pacing devices that employ atrial pacing and atrial sensing, detection and/or characterization of atrial activity is normally more difficult when compared to detection and/or characterization of ventricular activity. In addition, afterpotentials associated with atrial pacing further complicate detection and/or characterization of atrial activity. Thus, while various exemplary methods and/or devices presented herein optionally apply to ventricular activity, they may be particularly suitable for atrial activity. Thus, the description that follows, at times, references various atrial examples, understanding that ventricular analogs may also apply.

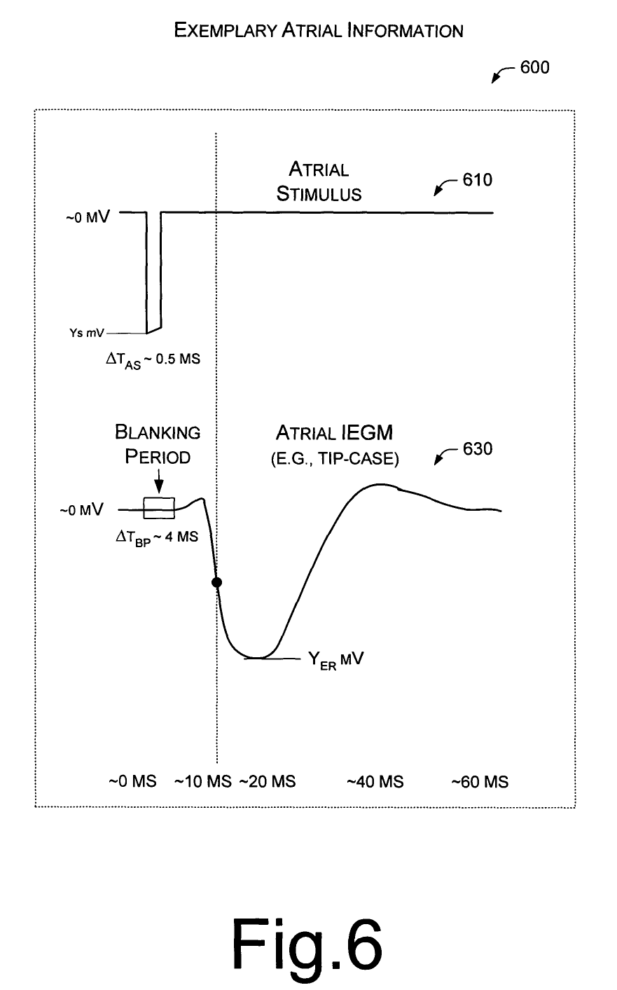

Referring to FIG. 6, exemplary atrial information 600 is shown. The exemplary atrial information 600 includes a plot of atrial stimulus voltage versus time 610 and a plot of sensed activity with respect to time 630. The exemplary time scale in the plot 630 illustrates that atrial activity typically occurs in a post-stimulus time frame of approximately less than one hundred milliseconds. Other events, such as the stimulation pulse width (e.g., Δt_{AS}˜0.5 ms) of the plot 610, are also exemplary and may not correspond precisely to the time scale of the plot 630.

The plot 610 exhibits a stimulus at approximately 0 ms, wherein the stimulus has a magnitude of Y_{S} mV and a duration of Δt ms. The atrial IEGM plot 630 exhibits an atrial stimulus at approximately 0 ms and a corresponding blanking period (e.g., Δt_{BP}˜4 ms).

As shown in FIG. 6, the IEGM in the plot 630 exhibits a minimum voltage, e.g., Y_{ER} mV, at approximately 20 ms. Just prior to this minimum (e.g., at approximately 10 ms), is a vertical dashed line and a solid dot that indicates one possible evoked response detection point. For example, a derivative-based evoked response detection scheme may detect a maximum negative derivative of voltage versus time at approximately the location of the dashed vertical line. Afterpotential may affect accuracy of such a derivative-besed evoked responce detection scheme. In particular, note that an afterpotential may have a negative slope in the neighborhood of the dashed vertical line and may also have a positive amplitude; whereas, the atrial IEGM has negative amplitude in the neighborhood of the dashed vertical line due to atrial depolarization. In instances where the positive amplitude of the afterpotential exceeds the negative amplitude of atrial depolarization, an atrial IEGM may actually have positive amplitude. While such a hypothetical atrial IEGM may, at such a neighborhood, have positive amplitude, it will typically maintain a negative slope. The presence of native activity may further complicate detection and/or characterization of “true” atrial activity. Thus, as described herein, techniques that account for afterpotential and/or more thoroughly characterize waveforms help in detection and/or characterization of cardiac activity.

Ensemble Averaging and/or Other Averaging

In ensemble averaging successive signals are collected and summed typically on a point-by-point or other basis. Therefore, a prerequisite for the application of ensemble averaging is the ability to reproduce a signal enough times to reach a desirable increase in signal to noise. Typical application of ensemble averaging is found in NMR and FT-IR spectroscopy, where the final spectrum is the result of averaging a plurality of individual spectra, which is often necessary to obtain a meaningful signal for cases where a single scan generates a practically unreadable signal heavily contaminated with random noise.

According to ensemble averaging, repetitive additions of noisy signals tend to emphasize their systematic characteristics and to cancel out any zero-mean random noise. If SNRₒ is the original signal-to-noise ratio of a signal, the final SNRf after N repetitions (or scans) is given by the following equation (Eqn. 1):

SNR_{f} = SNRₒ N^{0.5} (1)

| N | Increase in SNR |
|---|---|
| 2 | 1.4 |
| 4 | 2 |
| 6 | 2.4 |
| 8 | 2.8 |
| 10 | 3.2 |
| 25 | 5 |
| 100 | 10 |

Therefore, by averaging, for example, 10 (or 100, etc.) signals (e.g., signal data sets) an approximate 3-fold (or a 10-fold) reduction of noise level is achieved. Note that by increasing “N” from 10 to 25, the increase is SNR is approximately the same as increasing “N” from 2 to 10. Thus, the increase in SNR is greater for ensemble averaging the first 10 signals than for the adding of the next 15 to the average. For this reason, ensemble averaging is well-suited for use in implantable pacing devices wherein 10 signals are typically easily acquirable in less then aproximately 1 minute (e.g., consider a typical heart rate on the order of 60 beats per minute). As discussed herein, such ensemble averaging optionally provides for more robust detection and/or characterization of cardiac activity.

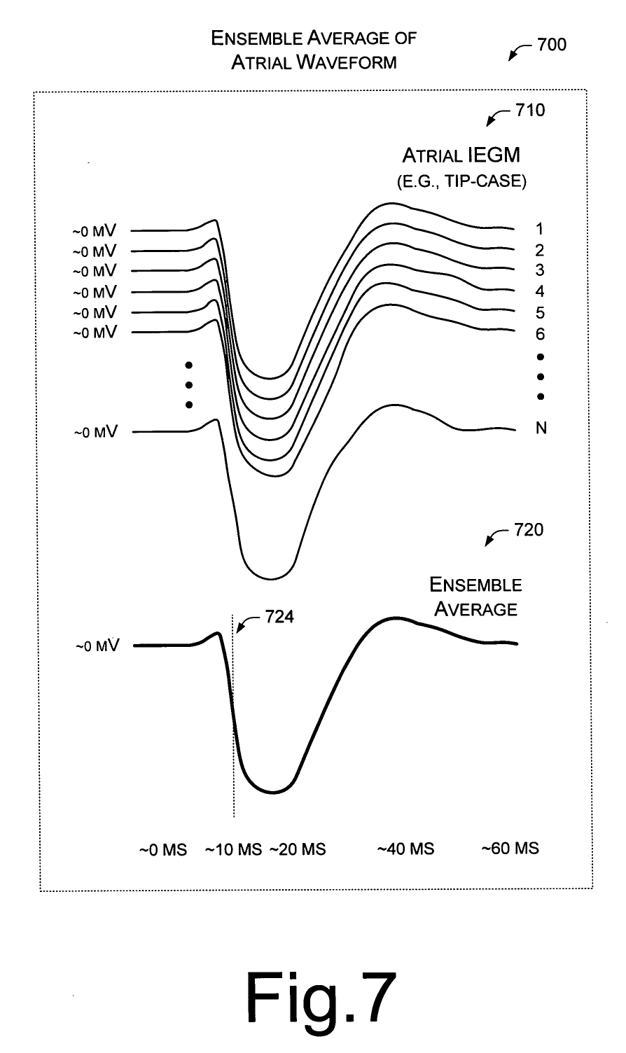

Referring to FIG. 7, an exemplary ensemble average scheme 700 for an atrial evoked response is shown. A series of atrial IEGMs (e.g., 1 to N) 710 are acquired responsive to successive atrial stimuli. In this example atrial stimuli are optionally delivered at a fixed rate and/or a variable rate that optionally depends on occurrence (or nonoccurrence) of another event. These individual atrial IEGMs 710 are the ensemble averaged to produce an ensemble average atrial IEGM 720. Of course, a criterion or criteria (e.g., standard deviation, etc.) are optionally used to exclude certain individual atrial IEGMs, for example, a noncapture atrial IEGM is optionally excludable. While variability between atrial IEGMs may,exceed variability between NMR scans, in general, an increase in SNR will be realized that can be approximated by Equation 1, above.

The ensemble average atrial IEGM 720 exhibits a vertical line at approximately 20 ms, which corresponds to a region wherein a derivative-based scheme may detect an atrial evoked response. In general, an ensemble average of atrial evoked responses (IEGM) may provide a base derivative value, a “window” and/or other information to aid in detection and/or characterization of atrial activity. Of course, a variety of other features exist within the ensemble average IEGM 720, which are optionally suitable for such purpose. Alternatively, or in addition to, the entire ensemble average (e.g., from approximately 10 ms or less to approximately 60 ms or more) is optionally suitable for use in detection and/or characterization of atrial activity. Note that as described below, timing and/or features may differ for a ventricular ensemble average.

Recognizing that the exemplary ensemble average 720 of FIG. 7 may contain afterpotential artifact, because afterpotential is typically not “random” noise, in some instances, analysis of afterpotential is optionally appropriate. For example, an exemplary ensemble average scheme for afterpotential acquires a series (e.g., 1 to N) of afterpotential responses (e.g., IEGMs) responsive to successive sub-threshold stimuli and/or stimuli delivered during a refractory period. In this example, the stimuli are optionally delivered at a fixed rate and/or a variable rate that optionally depends on occurrence (or nonoccurrence) of another event. Individual responses are then ensemble averaged to produce an ensemble average afterpotential response. Of course, a criterion or criteria (e.g., standard deviation, etc.) are optionally used to exclude certain individual afterpotential responses, for example, a capture IEGM is optionally excludable. While variability between IEGMs may exceed variability between NMR scans, in general, an increase in SNR will be realized that can be approximated by Equation 1, above.

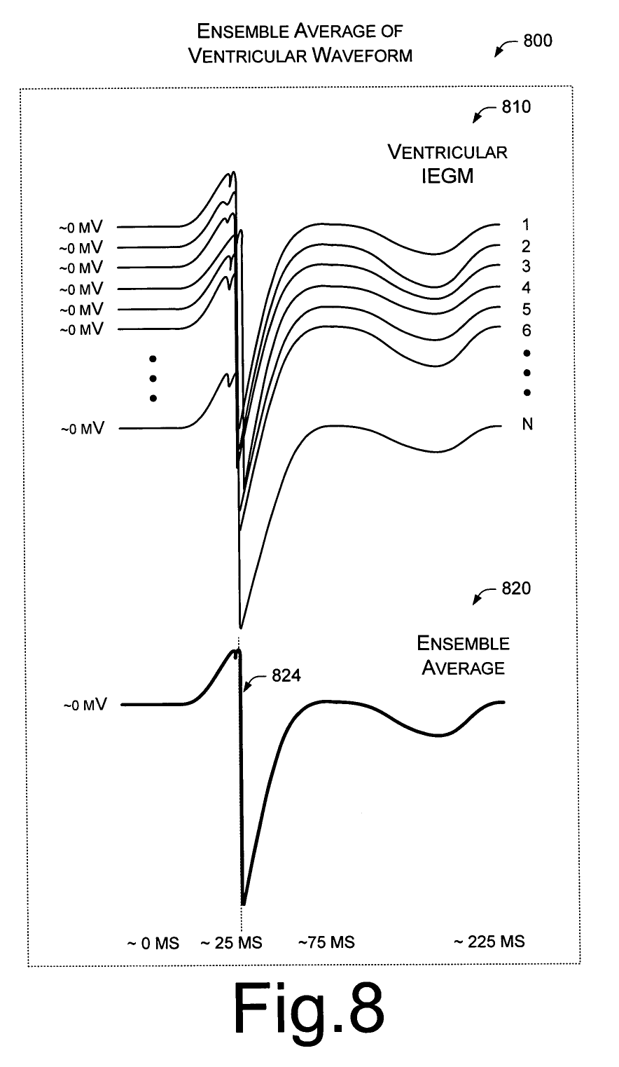

Referring to FIG. 8, an exemplary ensemble average scheme 800 for a ventricular evoked response is shown. A series of ventricular IEGMs (e.g., 1 to N) 810 are acquired responsive to successive ventricular stimuli. In this example, the ventricular stimuli are optionally delivered at a fixed rate and/or a variable rate that optionally depends on occurrence (or nonoccurrence) of another event. These individual ventricular IEGMs 810 are then ensemble averaged to produce an ensemble average ventricular IEGM 820. Of course, a criterion or criteria (e.g., standard deviation, etc.) are optionally used to exclude certain individual ventricular IEGMs, for example, a noncapture ventricular IEGM is optionally excludable. While variability between ventricular IEGMs may exceed variability between NMR scans, in general, an increase in SNR will be realized that can be approximated by Equation 1, above.

The ensemble average ventricular IEGM 820 exhibits a vertical line at approximately 100 ms, which corresponds to a region wherein a derivative-based scheme may detect a ventricular evoked response. In general, an ensemble average of ventricular evoked responses (IEGM) may provide a base derivative value, a “window” and/or other information to aid in detection and/or characterization of ventricular activity. Of course, a variety of other features exist within the ensemble average IEGM 820, which are optionally suitable for such purpose. Alternatively, or in addition to, the entire ensemble average (e.g., from approximately 10 ms or less to approximately 300 ms or more) is optionally suitable for use in detection and/or characterization of ventricular activity.

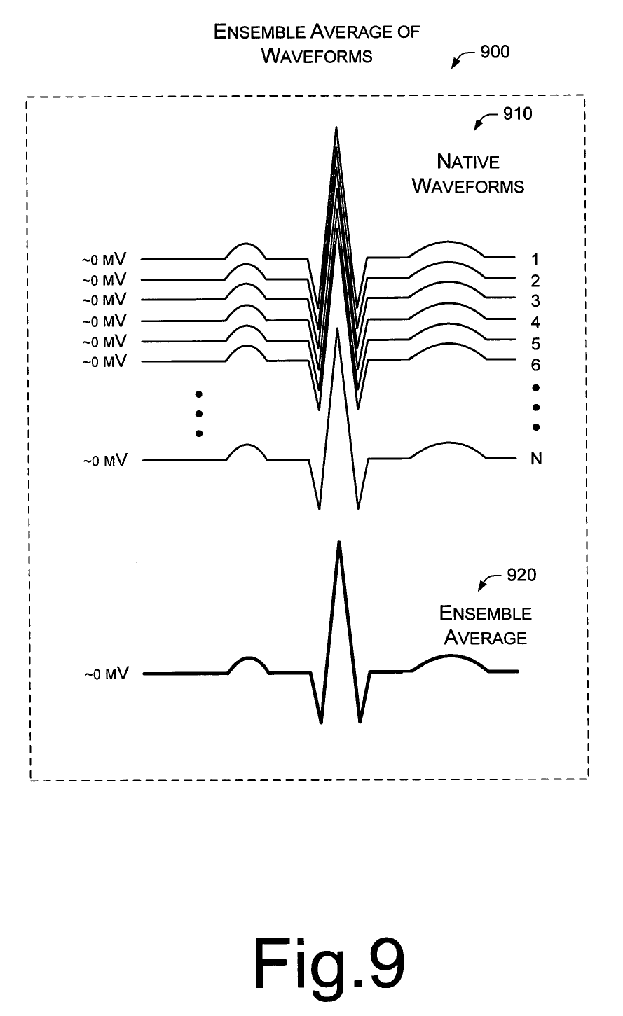

Referring to FIG. 9, an exemplary ensemble average scheme 900 for a native waveform is shown. A series of native waveforms (e.g., 1 to N) 910 are acquired responsive to successive native timing. These individual native waveforms 910 are then ensemble averaged to produce an ensemble average native waveform 920. Of course, a criterion or criteria (e.g., standard deviation, etc.) are optionally used to exclude certain individual native waveforms. While variability between native waveforms may exceed variability between NMR scans, in general, an increase in SNR will be realized that can be approximated by Equation 1, above.

The ensemble average native waveform 920 exhibits various features that correspond to a P wave, an R wave, a T wave, etc. A derivative-based scheme may suitably detect such waves. In general, an ensemble average of native waveforms may provide a base derivative value, a “window” and/or other information to aid in detection and/or characterization of cardiac activity. Of course, a variety of other features exist within the ensemble average 920, which are optionally suitable for such purpose. Alternatively, or in addition to, the entire ensemble average (e.g., from approximately 10 ms or less to approximately 500 ms or more) is optionally suitable for use in detection and/or characterization of cardiac activity.

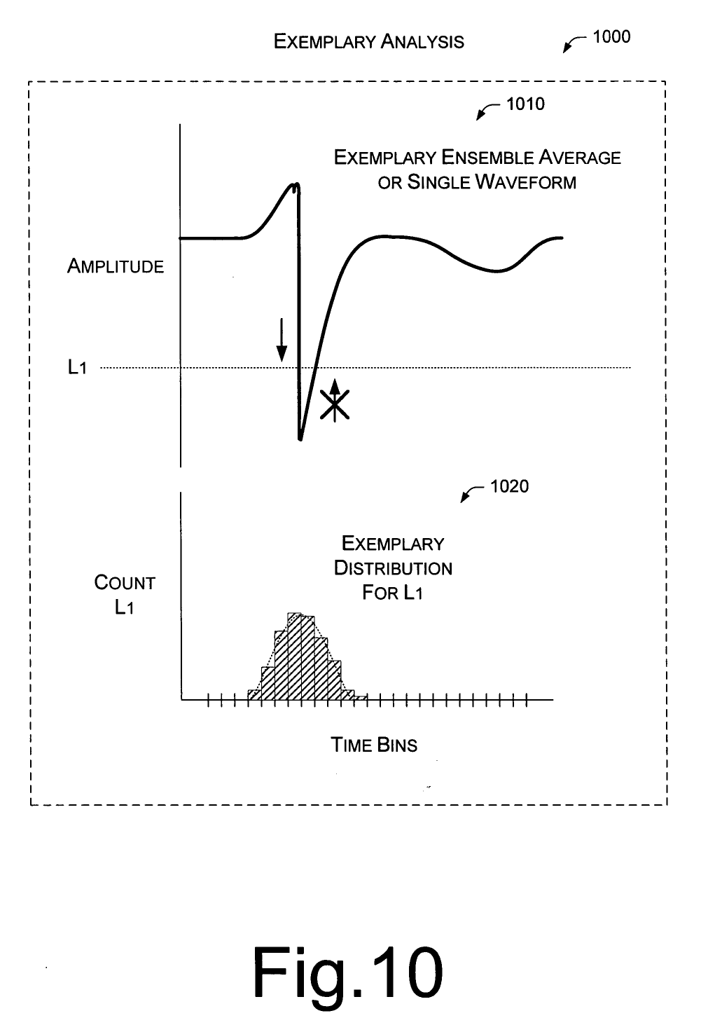

Referring to FIG. 10, an exemplary analysis 1000 is shown. The exemplary analysis 1000 includes an ensemble average or single waveform 1010 and an exemplary distribution 1020. The exemplary distribution 1020 includes various bins wherein each bin corresponds to a time or a time interval. According to the exemplary analysis 1000, a count is registered in the appropriate bin of the distribution 1020 where the amplitude of the ensemble average or the single waveform 1010 falls below a set level L1. Note that in this example, a count does not register where the amplitude of the ensemble average or the single waveform 1010 rises above the set level L1.

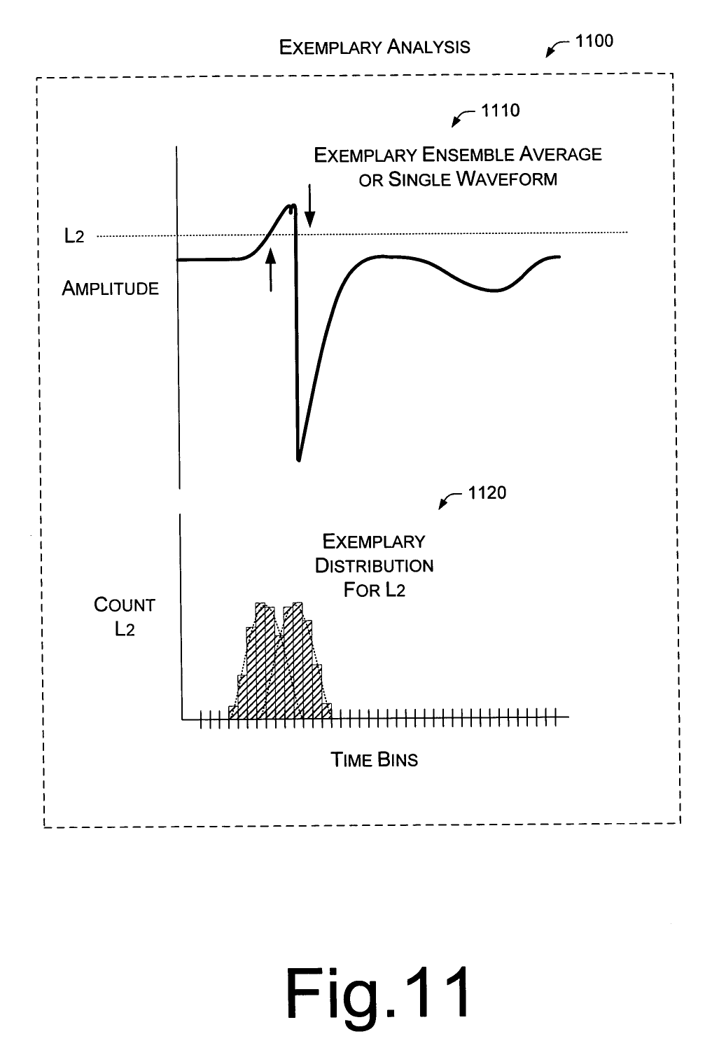

Referring to FIG. 11, an exemplary analysis 1100 is shown. The exemplary analysis 1100 includes an ensemble average or single waveform 1110 and an exemplary distributution 1120. The exemplary distribution 1120 includes various bins wherein each bin corresponds to a time or a time interval. According to the exemplary analysis 1100, a count is registered in the appropriate bin of the distribution 1120 where the amplitude of the ensemble average or the single waveform 1010 rises above or falls below a set level L2.

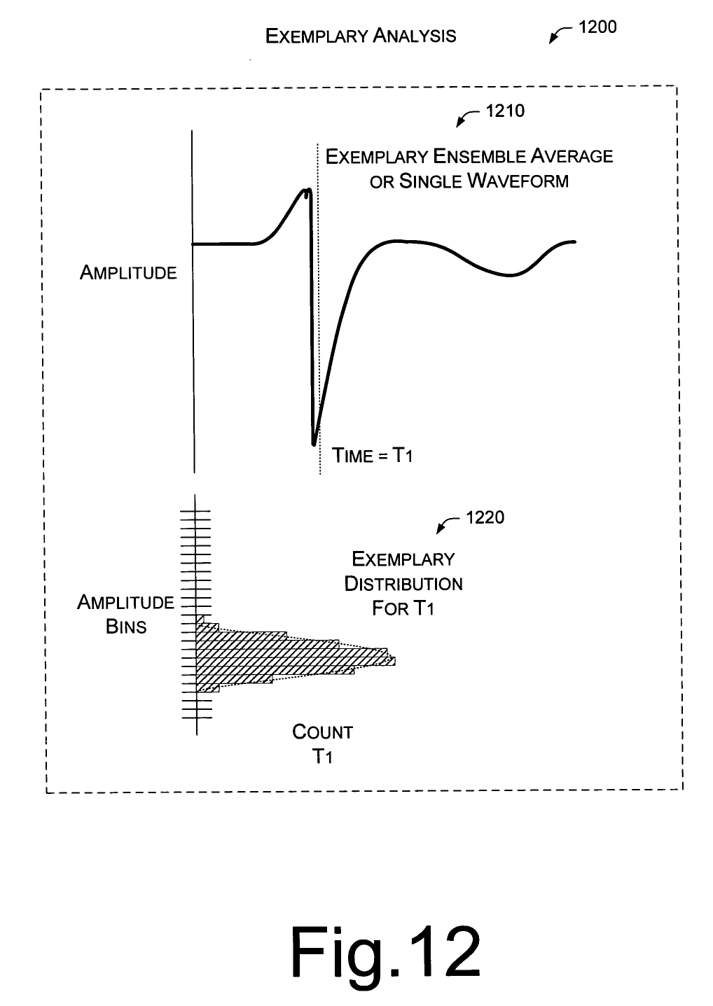

Referring to FIG. 12, an exemplary analysis 1200 is shown. The exemplary analysis 1200 includes an ensemble average or single waveform 1210 and an exemplary distribution 1220. The exemplary distribution 1220 includes various bins wherein each bin corresponds to an amplitude level or an interval. According to the exemplary analysis 1200, a count is registered in the appropriate bin of the distribution 1220 for amplitude at a set time T1. Of course, an alternative analysis optionally registers a count based on, for example, a minimum amplitude level, a maximum amplitude level, an amplitude level determined on the basis of a threshold and/or derivative technique.

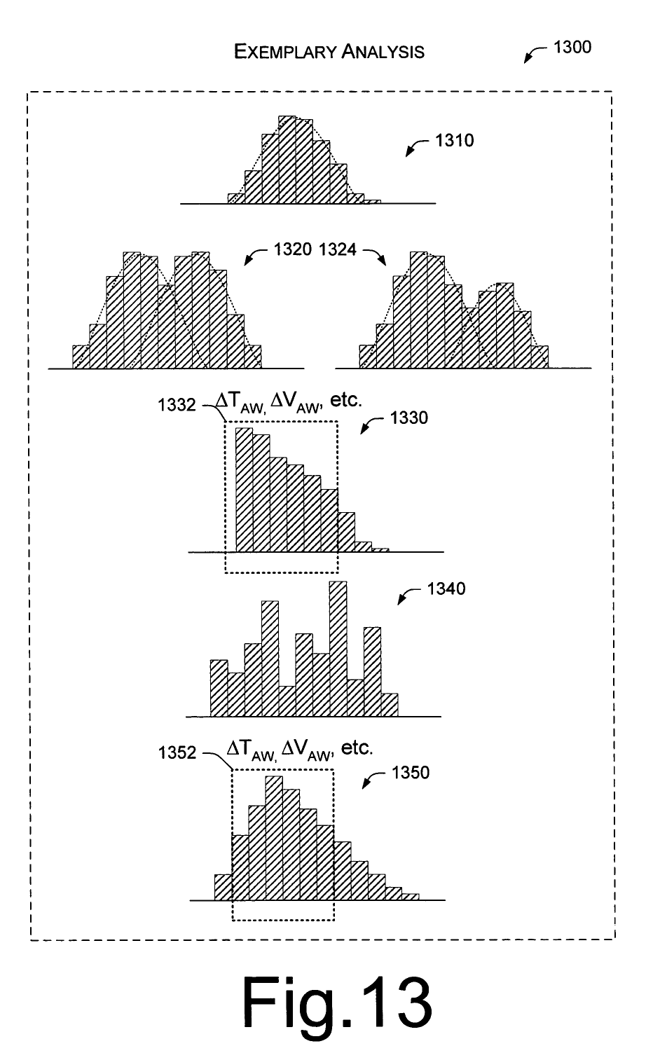

Referring to FIG. 13, various exemplary histogram distributions 1300 are shown. In general, histograms classify information into bins or regions for determining frequencies of certain events or categories of information. While the various exemplary histograms 1300 are shown as bar graphs, an implantable pacing device typically registers counts in one or more bins using one or more vectors, arrays, etc. Location, spread and/or shape of a histogram (or equivalent vector, array, etc.) optionally allow for further analysis of one or more processes related to generation of the underlying information. For example, a normal distribution, such as the exemplary distribution 1310, has a well known spread, shape and corresponding set of equations (e.g., statistical analysis equations, probability analysis equations, etc.). An analysis of such a histogram optionally focuses on a mean or a nominal value and/or a spread or variability of the information. For an implantable pacing device, a normal distribution of minimum amplitude level counts and/or other count information may indicate that the device is operating properly and/or that a patient's heart is operating properly.

An exemplary “bimodal” distribution 1320 includes two approximate peaks of relatively equal frequency. Such an exemplary distribution optionally results from an analysis such as the exemplary analysis 1100 of FIG. 11. A bimodal distribution may indicate that information corresponds to more than one underlying process. Another exemplary “bimodal” distribution 1324 includes two peaks of different frequency and/or other characteristics. Such frequency information may indicate a shift in an underlying process during information acquisition. For example, a shift in duration between an applied stimulus and an evoked response may produce a distribution such as the exemplary distribution 1324. In general, a frequency distribution does not record a time for each count; however, various exemplary methods and/or devices disclosed herein optionally record and/or otherwise track time for one or more counts. According to such an exemplary method and/or device, analysis of a multimodal distribution includes an ability to determine and/or associate a time or time average with one or more modes.

Yet another exemplary distribution 1330 exhibits a step down shape. Such a “single tailed” distribution may indicate a process limitation, for example, an event does not occur at a time less than that corresponding to the leftmost filled bin. The right-side tail may indicate that an abrupt limit does not exist for times greater than that corresponding to the leftmost filled bin. Of course, the exemplary distributions 1310, 1320, 1324 optionally exhibit tails having different characteristics and/or one or more skewed distributions. In addition, an acquisition and/or analysis window 1332 having a width of Δt_{AW}, ΔV_{AW}, etc., is also shown (e.g., corresponding to a time window, a voltage window and/or another parameter window). The beginning, the end, and/or the duration of the window 1332 are optionally selected to facilitate analysis and/or to acquire information over a specific period of time. The exemplary window 1332 does not coincide with the distribution data 1330 and, thus, may represent an analysis window rather than an acquisition

An exemplary comb distribution 1340 has a shape or pattern that does not readily appear to include an underlying normal, bimodal or other process. Such a distribution may indicate interference by noise and/or another problem. An exemplary skewed distribution 1350 has tails that differ. Again, such a distribution may indicate that an underlying process has asymmetric characteristics. A comparison of the two tails may aid in an analysis and, for example, in setting pacing parameters to ensure suitable performance. In addition, an acquisition and/or analysis window 1352 having a width of Δt_{AW,} ΔV_{AW,} etc., is also shown (e.g., corresponding to a time window, a voltage window and/or another parameter window). The beginning, the end, and/or the duration of the window 1352 are optionally selected to facilitate analysis and/or to acquire information over a specific period of time. The exemplary window 1352 does include all of the distribution data 1350 and, thus, may represent an analysis window rather than an acquisition window. An acquisition window would typically coincide with limits of a distribution.

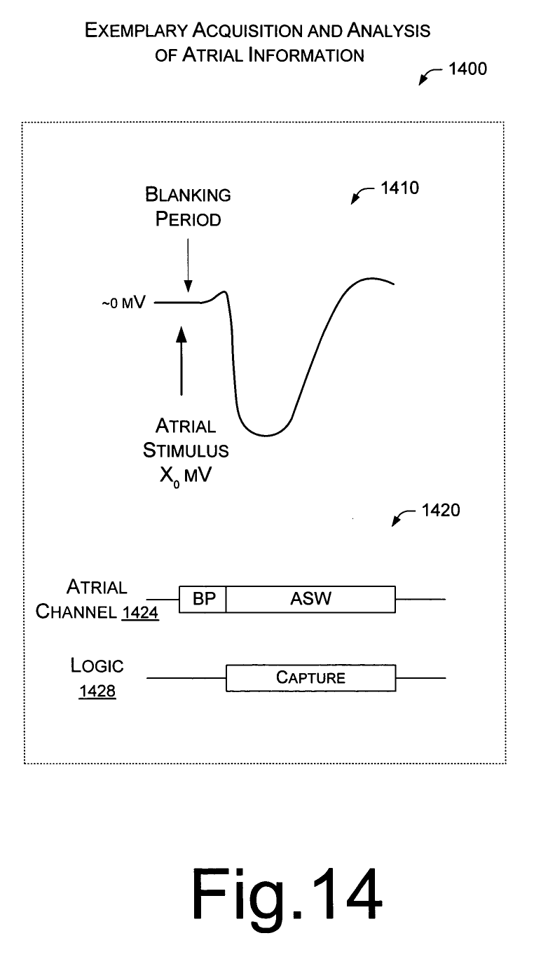

Referring to FIG. 14, an exemplary method 1400 for acquisition of an evoked response is shown. An exemplary waveform 1410 exhibits an atrial stimulus (X_{O}) followed by a blanking period and an atrial evoked response. An atrial channel 1424 also shows these events as a blanking period (BP) and an atrial sensing window (ASW). Of course, the duration of the blanking period and/or sensing window may vary and/or be discontinuous in that they do not occur precisely back-to-back as shown. A logic line 1428 shows logic corresponding to the IEGM 1410 and the atrial channel 1424. For example, the logic 1424 indicates capture based on analysis of information acquired during one or more ASWs. Such an analysis optionally uses ensemble averaging and/or histogram analysis (or other suitable statistical analysis). In addition, in analyzing information acquired as shown in FIG. 14, an implantable pacing device optionally subtracts an afterpotential from a prior acquired IEGM that includes an afterpotential artifact to produce an artifact removed result on a beat-by-beat and/or other basis. One or more artifact removed results are optionally ensemble averaged.

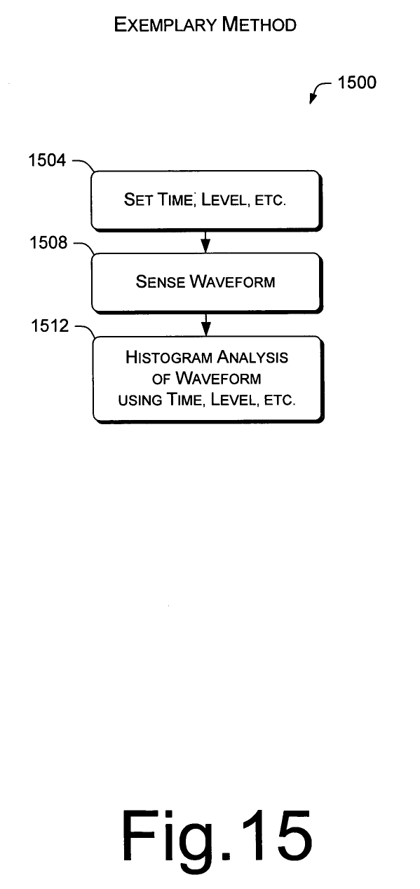

Referring to FIG. 15, an exemplary method 1500 for histogram analysis is shown. In a set block 1504, an implantable pacing device sets a time, a level or other parameter to allow the device to discern an event. Next, in a sense block 1508, the implantable pacing device senses a waveform usually as waveform information. During and/or after sensing, in a histogram analysis block 1512, the implantable pacing devices uses histogram analysis to analyze the waveform, for example, using the set time, level or other parameter to discern an event and register a count for the event. In general, the implantable pacing device registers the count in an appropriate bin. The analysis optionally includes an analysis of counts in one or more bins to enhance pacing therapy, such as, but not limited to, enhancing pacing therapy via changing one or more pacing parameters in response to the analysis.

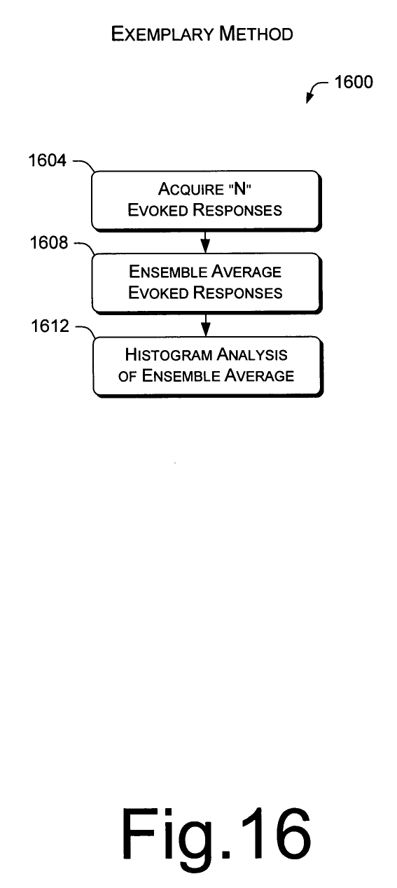

Referring to FIG. 16, an exemplary method 1600 is shown for ensemble averaging and histogram analysis. In an acquisition block 1604, an implantable pacing device acquires N₀ evoked responses (e.g., IEGMs). Next, in an ensemble average block 1608, the device determines an ensemble average of the N₀ evoked responses. Of course, such a block optionally operates simultaneous with the acquisition block 1604 wherein an average is determined on an evoked response-by-evoked response basis. A histogram analysis block 1612 follows wherein a histogram analysis of the ensemble average occurs. Of course, the histogram analysis optionally occurs in conjunction with one or more analyses. Ensemble averaging of waveforms prior to histogram analysis optionally acts to reduce noise and facilitate subsequent analysis. For example, a histogram based on individual sensed waveforms may exhibit unacceptable noise. While such noise is potentially minimized by decreasing the number of bins (e.g., increasing bin width), important details may be lost. Thus, an exemplary method (e.g., the method 1600) optionally uses ensemble averaging to minimize noise and/or produce a template suitable for use in one or more other analyses, and then, uses histogram analysis to register counts in bins. Such a method may allow for finer bins, which, in turn, may allow for detection and/or characterization of finer details contained in a waveform. An exemplary method using ensemble averaging optionally averages from approximately 3 to approximately 100 waveforms. In turn, according to this example, an implantable pacing device generates a histogram based on approximately 5 to approximately 1000 ensemble averages.

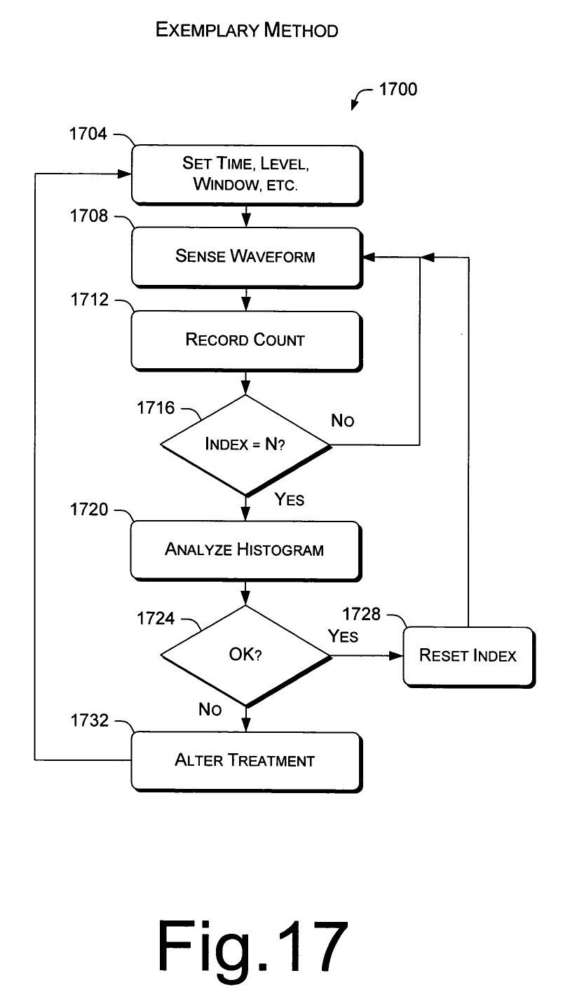

Referring to FIG. 17, an exemplary method for pacing therapy is shown. In a set block 1704, an implantable pacing device sets a time, a level, a window and/or other parameter(s) to allow the device to discern an event. Next, in a sense block 1708, the implantable pacing device senses a waveform usually as waveform information. During and/or after sensing, in a record block 1712, the implantable pacing device uses the setting of the set block 1704 to discern an event and to record the occurrence of the event as a count. A decision block 1716 follows wherein the device determines whether an index limit has been reached, for example, whether an index has reached a limit “N” (e.g., where N is an integer, etc.). If the decision block 1716 determines that the index has not reached the limit, then the exemplary method 1700 continues in the sense block 1708. Of course, other action may occur prior to the sense block 1708, such as, but not limited to, a pacing stimulus.

If the decision block 1716 determines that the index reached a limit, then the method 1700 continues in an analysis block 1720 capable of performing histogram analysis. The analysis block 1720 analyzes the recorded N counts to detect and/or characterize cardiac function and/or device function. For example, the analysis optionally detects normal or abnormal cardiac function or normal or abnormal device function.

Following the analysis block 1720, another decision block 1724 determines whether the analysis calls for a change in pacing therapy and/or patient therapy. If the decision block 1724 determines that a change is necessary and/or desirable, then in an alteration block 1732, the implantable pacing device alters therapy and/or alerts a patient, physician, etc., to alter therapy. The alteration block 1732 and/or the decision block 1724 optionally return to the set block 1704. If the decision block 1724 determines that no change is required and/or desired, then the method 1700 may continue at the sense block 1708. Of course, other action optionally occurs prior to sensing at the sense block 1708.

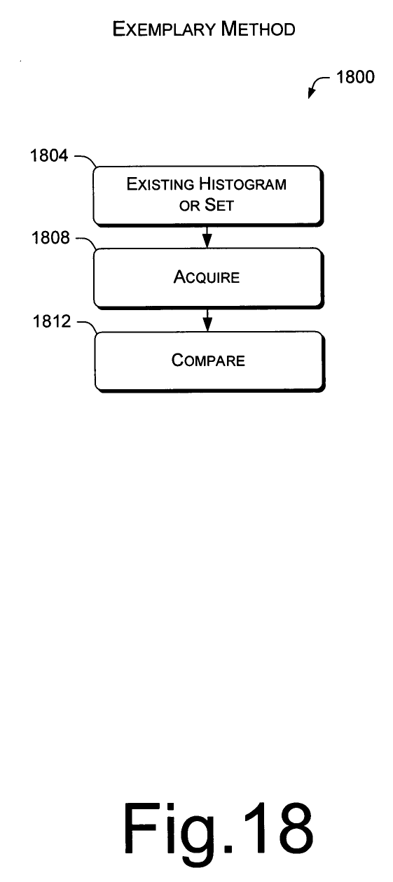

FIG. 18 shows an exemplary method 1800 for comparing acquired information to existing information. In a selection block 1804, an existing data set, model, and/or histogram are selected. Next, in an acquisition block 1808, information is acquired. A comparison block 1812 then compares the acquired information to the existing information. The result of the comparison is optionally used to alter cardiac therapy, to select new existing information, to acquire new information and/or perform a new comparison.

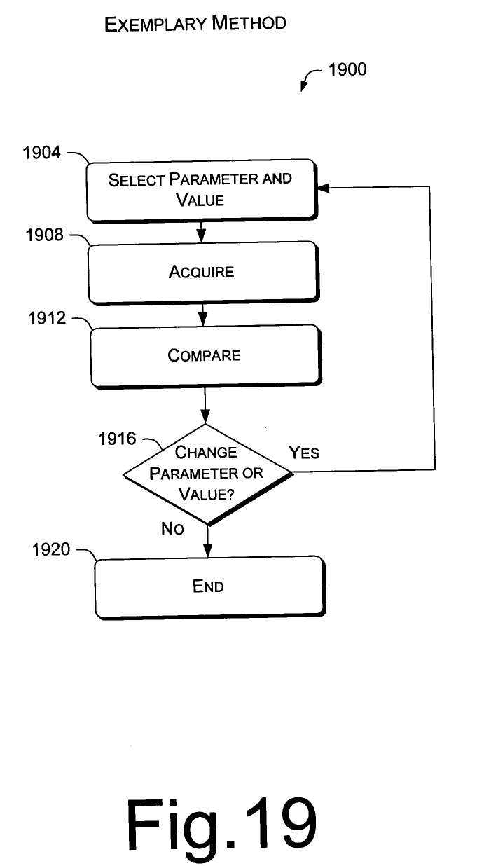

FIG. 19 shows an exemplary method 1900 for acquiring and comparing information based on one or more physiologic parameters. In a selection block 1904, a particular physiologic parameter is selected (e.g., an atrial or a ventricular pacing rate, etc.) along with a measured or set value for the parameter. Next, in an acquisition block 1908, information is acquired. The acquired information may be in the form of ensemble averages and/or histograms associated with ensemble averages. A comparison block 1912 follows that compares the acquired information to previously acquired information having some association with the selected physiologic parameter. For example, if the selected parameter is a pacing rate of 60 beats per minute, then the previously acquired information may correspond to acquired information associated with a pacing rate of 70 beats per minute. The comparison may reveal differences in various intrinsic or induced waves (e.g., A, Q, R, S, T, etc.), in morphology of such waves (e.g., fusion, pseudofusion, capture, etc.) and/or in timing of such waves (e.g., intervals, width, etc.). Of course, the previously acauired information may be associated with a pacing rate of 60 beats per minute to elucidate other non-rate related physiologic changes via a comparison of ensemble averages and/or histograms. After the comparison block 1912, a continuation block 1916 optionally follows wherein additional information may be acquired for the same parameter value or for a different parameter or a different parameter value via a return to the selection block 1904. The exemplary method 1900 terminates in an end block 1920 if the case that no further comparison of parameters or values is desired.

### Conclusion

Although exemplary methods and/or devices have been described in language specific to structural features and/or methodological acts, it is to be understood that the subject matter defined in the appended claims is not necessarily limited to the specific features or acts described. Rather, the specific features and acts are disclosed as exemplary forms of implementing the claimed methods and/or devices.

1. A method for analyzing an intracardiac electrocardiogram in an implantable cardiac device, the method comprising:
acquiring a plurality of intracardiac electrocardiogram signals;
ensemble averaging the plurality of intracardiac electrocardiogram signals to produce an ensemble average;
repeating the acquiring and ensemble averaging one or more times to produce a plurality of ensemble averages; and
processing the plurality of ensemble averages to generate a model of cardiac activity.

2. The method of claim 1, wherein processing comprises detecting an event within each ensemble average using one of a time and a level.

3. The method of claim 1, wherein acquiring comprises acquiring a continuous intracardiac signal and splitting the continuous intracardiac signal into the plurality of intracardiac electrocardiogram signals.

4. The method of claim 1, wherein processing comprises generating a histogram based on information from the plurality of ensemble averages.

5. The method of claim 4, wherein the histogram represents counts versus time.

6. The method of claim 4, wherein the histogram represents counts versus amplitude.

7. The method of claim 2, wherein the event is an evoked response.

8. The method of claim 2, wherein the event is an atrial event.

9. The method of claim 2, wherein the event is a ventricular event.

10. The method of claim 4, wherein the histogram represents occurrence of ventricular and/or atrial events with respect to time.

11. The method of claim 4, wherein the histogram represents occurrence of ventricular and/or atrial events with respect to amplitude.

12. The method of claim 1, wherein the repeating occurs for at least approximately 5 times.

13. The method of claim 1, wherein the repeating occurs from approximately 10 to approximately 100 times.

14. The method of claim 4, further comprising using the histogram to alter cardiac therapy.

15. The method of claim 4, further comprising analyzing the histogram to characterize cardiac function as normal or abnormal.

16. The method of claim 1, further comprising comparing an intracardiac electrocardiogram to the ensemble average.

17. The method of claim 4, further comprising analyzing an intracardiac electrocardiogram using the histogram.

18. An implantable cardiac device comprising:
means for acquiring intracardiac electrograms corresponding to a plurality of events;
means for generating an ensemble average for each of the plurality of sets of intracardiac electrograms; and
means for processing the plurality of ensemble averages to generate a model of cardiac activity.

19. The device of claim 18, further comprising means for comparing an ensemble average to information contained in an intracardiac electrocardiogram.

20. The device of claim 18, further comprising means for analyzing an intracardiac electrocardiogram using the histogram.

21. An implantable cardiac device for analyzing an intracardiac electrocardiogram in an implantable pacing device, the device comprising:
a lead system operative to acquire an intracardiac electrocardiogram signal;
a processor connected to the lead system and operative to ensemble average a plurality of cardiac events within the intracardiac electrocardiogram signal, wherein the processor is operative to use the ensemble average to classify subsequent events acquired by the lead system.
